(19) United States
(12) Patent Application Publication
(10) Pub. No.: US 2004/0133131 A1
(43) Pub. Date: Jul. 8, 2004

(54) In vivo ruminant health sensor

(76) Inventors: David L. Kuhn, Cave Creek, AZ (US); Daniel Kohn, Cave Creek, AZ (US)

Correspondence Address: Dr. David L. Kuhn, P.O. Box 7085, Cave Creek, AZ, 85327, US

(21) Appl. No.: 10/336,062

(22) Filed: Jan. 3, 2003

(51) Int. Cl. A61B 5/103

(52) U.S. Cl. 600/593

(57) ABSTRACT

A system and method of determining a physiological state of a ruminant animal by monitoring the pH and temperature of the animal's stomach. A battery-less, single unit sensor and transmitter is placed within the rumen or reticulum of the animal. pH and temperature measurements are taken and transmitted along with the animal identification code to a wireless receiver. The physiological state of the animal is determined using the mathematically analyzed pH and temperatures.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

Ruminant animals contain four stomach compartments called the reticulum (reticulorumen), the rumen, the omasum, and the abomasum. All four stomachs are used for the digestion of foodstuffs; however, digestion by microorganisms takes place only in the reticulum and the rumen, the first two stomachs. The reticulum is the largest of a number of sacs that comprise the rumen.

The high-surface area, honeycomb structure of the reticulum walls act to mechanically impede the passing of hardware (indigestible matter, such as nails, wire, or rocks) into the remaining digestive tract. The reticulum contains up to 3 gallons of digesta. Food from the reticulum may be regurgitated as the cud. Many of the dairy cows in the United States have been outfitted with a rumen magnet, introduced orally to the reticulum, which prevents swallowed metallic hardware from being regurgitated. The action of regurgitation increases the likelihood of puncture, infection, and subsequent serious tissue damage. This is often referred to as “Hardware Disease”.

The rumen, in a mature cow, nearly fills the entire left side of the abdominal cavity and can contain 40-60 gallons of digesta. The digesta of the rumen is loaded with approximately 10¹¹ microbes per cc, consisting of bacteria, fungus, and protozoa. Each of the many types of microbes performs different digestive functions. Furthermore, each of the microbe types thrives under different temperature and pH conditions. For proper digestion to occur in the rumen, the temperature must be maintained between 100 and 108 degrees Fahrenheit and the pH maintained within the 5.8 to 6.4 range.

Deviations in proper pH and temperature within the rumen can have many causes, including improper diet and infection. Detection of persistent deviation can therefore be used as a preventative tool for diagnosing and removing diseased cows from the herd. Persistent deviations can also lead to many subsequent and undesirable results, including development of low pH resistant bacteria that is passed to the milk. Numerous veterinary studies have been performed documenting relationships between pH, temperature and cow health.

Many of the cows in the United States have been outfitted with a uniquely identifiable transponder. Farmers detect the presence of the cow during feeding or milking time when the cow's transponder comes within range of the primary receiving antenna. Once detected, the presence of the cow can be registered in a database for tracking. Databases can be manually or automatically queried to detect missing cows. Transponders are typically worn as a necklace, stapled to the ear, or injected subcutaneously. Recent FDA regulations, however, prohibit the introduction of transponders in beef cow tissue that can become mixed with meat during slaughter.

An Electronic Animal Identification System was described by U.S. Pat. No. 5,482,008 [Stafford and Kilroy, Jan. 9, 1996]. Their bolus was designed to be swallowed by a cow and contained an identification tag and transponder that could transmit the Cows id tag to a receiver. U.S. Pat. No. 5,984,875 [Brune Nov. 19, 1999] and U.S. Pat. No. 6,099,482 [Brune, et al Aug. 8, 2000] describe a bolus device that contains an identification tag, a temperature sensor, electronics, and a battery that can collect and transmit the identification and temperature to a receiver. U.S. Pat. No. 6,371,927 [Brune, et al Apr. 15, 2002] further describes technology to sense and transmitting physiological parameters and U.S. Pat. No. 6,059,733 [Brune et al May 9, 2000] describes a method for determining a physiological state of a ruminate animal by monitoring the core body temperature.

Accordingly, the objects or advantages of this invention are to provide an inexpensive, reliable, safe, and long lasting device to collect data on the pH and temperature in a ruminant's reticulum and to transmit that data to a receiver for processing and posting to the cow's health record. Further object will become apparent from a consideration of the ensuing description and drawings.

### BRIEF SUMMARY OF THE INVENTION

The preferred embodiment of the invention is packaged in a small container, 2 cm or greater in length and 1 cm or greater in width. In the preferred embodiment, the device is given orally to a cow and comes to rest in the reticulum where it may associate with the rumen magnet.

In the preferred embodiment of the invention a thermistor, or other temperature-measuring device, is used to measure the ambient temperature of the cow's reticulum. The temperature value is digitized and stored in memory.

In the preferred embodiment of the invention a galvanic device is used to measure pH The device produces an electrical potential between two leads as a result of the concentration of ambient hydronium ions in the cow's reticulum. The voltage is digitized and stored in memory.

In the preferred embodiment, the transponder tag and associated antenna components of the device communicate with a receiver and its associated antenna. In the preferred embodiment, the penetration of the radio signals through the aqueous environment of the cow's stomach will be impeded to a minimal extent. Also, the signal should be as insensitive to antennae orientations as possible. Radio frequencies are chosen accordingly.

In the preferred embodiment of the invention, the transponder tag communicates the tag ID number with the receiver. In the preferred embodiment, the tag ID, temperature and pH readings are all communicated with the receiver at the same time, as a single digital sequence of numbers.

In the preferred embodiment, using standard transponder technology, a single receiver communicates with multiple tags. Communication occurs when the tag is within range of the receiver. In the preferred embodiment of the invention, the receiver antenna is located at milking or feeding stations, although hand-held portable receivers are also employed. In the preferred embodiment, the receiver differentiates between multiple simultaneous tag signals.

In the preferred embodiment of the invention, the receiver communicates the data from the tags with a remote or local computer and associated software. The software is able to remove the tag ID, pH voltage, and temperature voltage from digital sequence and convert all voltages to corresponding actual pH and temperature values.

In the preferred embodiment, the ID, pH, and temperature values are stored in a database, which is a component of the associated software. The database is queried automatically at regular intervals. The preferred embodiment of the invention will be used for preemptive diagnostic and health tracking purposes. Triggers, such as low/high temperature or pH aberrations and trends, which can be stored in DB and used in the automatic DB query can produce alert condition where appropriate. Alert conditions can be stored in the DB and/or sent via other electronic means to a pager or telephone.

### DESCRIPTION OF DRAWINGS

FIG. 1 is a schematic representation of the physical layout of the device, including all physical components and packaging.

FIG. 2 is a schematic representation of the electronic circuit and components used in the device. The schematic includes all components used to measure pH, temperature, and optionally additional physical or chemical values and transmit them from the device to a receiver using standard transponder technology.

### REFERENCE NUMERALS IN DRAWINGS

100 pH Sample Probe

200 pH Reference

300 Temperature sensor

400 Multiplexer

500 Analog to Digital Converter

600 Serializer

700 Sequencer

800 ID tag

900 Power Good Enable

1000 Antenna

1100 Metallic Backing

1200 Casing

### DETAILED DESCRIPTION OF INVENTION

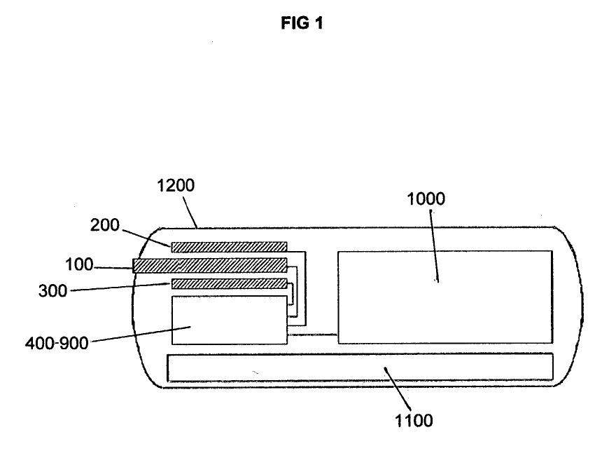

FIG. 1 provides a high level overview of the physical components that comprise the device. The preferred embodiment of the invention includes a metallic mass (1100) on one face of the device that will serve to attract the device to the reticulum magnet so that it neither can be regurgitated nor passed out through the remaining digestive track. Also, the preferred embodiment of the invention includes an inert coating (1200) surrounding the device. The material may be thin Teflon, silicate, or other surface.

In the preferred embodiment, the pH probe (100) extends beyond the boundary of the inert coating whish encompasses the rest of the device. This physical extension is required in order to expose the galvanic material to the hydronium ions within the reticulum of rumen fluid.

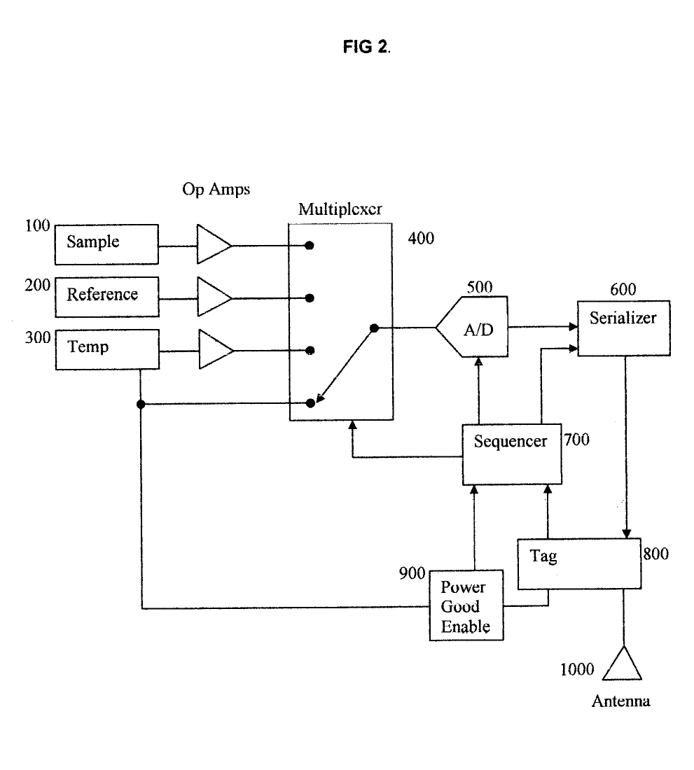

FIG. 2 provides a high level overview of the electronic components and the physical devices that provide the measurements to be transmitted. In the preferred embodiment, the invention is used to measure the pH of the fluid in which the device resides. The sample probe (100) contains standard galvanic elements that are sensitive to hydronium ion concentrations. The exposed end of the sample probe, see FIG. 1, contains a glass or other material that is integral to the component. Such galvanic devices have known linear voltage response curves to pH and temperature. At room temperature, a change of 1 pH unit causes a voltage change of about 60 millivolts, or 0.060 volts. At 0 degrees centigrade, 1 pH unit causes a 54 mV change. At 100 degrees centigrade, a 1 pH unit change causes a 70 mV change. At pH of 7, the voltage generated is 0 mV. The device will need to measure the healthy range of a cow pH, 5.8-6.4, as well as aberrant values that may range from 4-8. Consequently, the device will generate from 0.180 V on the low pH range and 0.060 V on the high pH range, the voltages being inverted at a pH of 7.0.

In the preferred embodiment, the invention will include a reference sample (200) of a solution of known pH, whose voltage output may be used as a comparison against the sample voltage. The reference sample solution contains a galvanic device made from the same material as the sample probe.

Both sample and reference voltage outputs can be conditioned. Conditioning includes signal amplification using an op-Amp, or similar device. In FIG. 2, the op-Amps are positioned between voltage source and the multiplexer (400). Additional or alternate conditioning may be achieved with an op-Amp or similar device located between the multiplexer (400) and the A/D converter (500).

In the preferred embodiment, the device includes a component (300) for measuring the temperature of the fluid in which it is located. This component may be a thermistor, thermocouple, or other device. In the preferred embodiment, the device will be used to measure and report temperature readings many times a day. Given this frequency, the thermocouple or other temperature device will be at thermal equilibrium with the surrounding solution to within acceptable tolerance, without making direct physical contact with the fluid mass. In the case of the thermistor device, an input voltage will pass through the component, resulting in an output voltage that is dependent upon temperature. The output voltage from the temperature device will also pass through an op-amp in order to amplify the signal.

In the preferred embodiment, the voltage output from the op-amps or other signal conditioning circuitry will go into a multiplexer (400). We describe the operation of the multiplexer in the text below. The multiplexer selects which input undergoes A/D conversion based on ourput from the sequencer (700) described below.

In the preferred embodiment, the transponder tag (800) is powered by rectifying the incoming RF carrier signal that is transmitted from the reader. When the tag develops sufficient DC voltage, it transmits the content of its memory array by modulating the RF carrier signal. In the preferred embodiment, the invention will use this DC power supply to provide the voltage needed for all components of the device. Alternate embodiments, where the power generated by the tag is augmented by a secondary power source, could use a piezoelectric device or the galvanic voltage generated by the hydronium ion concentration.

The circuitry of the device is designed to enable the tag to accept output from any number of sensors regardless of the number of external inputs available on the tag. The invention uses a serializer (600) to string together the multiple digital outputs of the multiplexer into a single digital string. The single string is output to the tags external input pin(s). The serializer may be constructed from a programmable logic device, a field programmable gate array, or other electronic devices. If a tag containing multiple external input channels is used, the serializer may not be required. The modulated RF carrier signal includes the tag ID as well as the output from the serializer.

In the preferred embodiment, the second input to the sequencer is a clock signal. The clock signal can come from the tag or, if one is not present as a pin in the tag, from a separate clock contained in the circuit of the invention. Provide the logical state from the power good enable (900) is true, the sequence uses the clock signal to instruct the multiplexer to alternate connections from the analog-to-digital device (500) to the four different input voltages to the multiplexer. The sequencer signal is also used by the A/D device to coordinate the transfer of the digitized voltage outputs into the appropriate output channels for passage to the serializer. Furthermore, the clock signal from the sequencer is also used by the serializer to coordinate the serialization of the difital bits corresponding to the four voltage inputs of the multiplexer.

In the preferred embodiment, the output from the serializer can contain the four digitized values, each as a 16-bit string, totaling 64 bits. The tag adds a unique ID to the digital string prior to transmission to the receiver.

In the preferred embodiment, the signal from the serializer is passed to the transponder tag and stored in its memory array. In the preferred embodiment, the tag transmits the content of its memory array to the receiver by modulating the incoming RF carrier signal.

In the preferred embodiment, the RF signal received by the receiver is converted back to the digital string input to the tag from the serializer plus the digital value of the tag ID itself. These two strings are sent from the receiver to a computer for further mathematical processing.

We claim:

1. An in-vivo sensor device for ingestion and retention in the rumen or reticulum of a ruminant animal, comprising:
a wireless transmitter and associated electronics including antenna and memory;
temperature and pH sensors;
encapsulated in an inert material forming single, battery-less unit,
powered by the said internal pH sensor and inductance from a remote interrogator through said antenna.

2. The device of claim 1, wherein encapsulation material is epoxy.

3. The device of claim 1, wherein encapsulation material is ceramic.

4. The device of claim 1 wherein encapsulation material may be coated with Teflon.

5. A method for determining a physiological state of a ruminant animal by monitoring the pH of the contents of the rumen or reticulum comprising the steps of:
providing a bolus within the rumen or reticulum of the ruminant animal, said bolus including a pH sensor and a transmitter;
sensing the pH within the stomach using said sensor;
transmission of air-borne signal from the bolus to a remote receiver using said antennae, the signal representing said pH;
mathematically analyzing one or more of said pH measurements;
determining the physiological state of the ruminant animal using said mathematically analyzed pH measurements.

6. The method of claim 5, wherein said mathematical analysis step includes the substep of buffering multiple pH measurements in volatile or persistent electronic data format.

7. The method of claim 6, wherein said mathematical analysis step comprises the substep of calculating the mathematical difference between said pH measurement and at least one threshold value.

8. The method of claim 6, wherein said mathematical analysis step comprises the substep of calculating the difference of two or more pH measurements.

9. The method of claim 8, wherein said mathematical analysis step comprises the substep of dividing the said difference of two pH measurements by the length of the time interval between the two measurements.
